(19) United States
(12) Patent Application Publication
(10) Pub. No.: US 2006/0212063 A1
(43) Pub. Date: Sep. 21, 2006

(54) Surgical device and associated trans-organ surgical method

(75) Inventor: Peter J. Wilk, New York, NY (US)

Correspondence Address: COLEMAN SUDOL SAPONE, P.C., 714 COLORADO AVENUE, BRIDGE PORT, CT, 06605-1601, US

(73) Assignee: WILK PATENT, LLC, New York, NY (US)

(21) Appl. No.: 11/369,616

(22) Filed: Mar. 7, 2006

Related U.S. Application Data
(60) Provisional application No. 60/662,980, filed on Mar. 18, 2005.

(51) Int. Cl. A61M 29/00

(52) U.S. Cl. 606/191

(57) ABSTRACT

A tubular member is inserted into a natural body cavity such as the vagina or colon for retracting that internal organ to facilitate surgical access to the abdominal cavity through a wall of the organ. An end closure is formed with an aperture for the insertion of medical instruments. The tubular member is provided with a clamping element at the one end for holding the tubular member to the wall of the organ about an artificial opening incised into the organ wall.

### CROSS-REFERENCE TO RELATED APPLICATION

This application claims the benefit of U.S. Provisional Patent Application No. 60/662,980 filed Mar. 18, 2005.

### BACKGROUND OF THE INVENTION

This invention relates to medical procedures carried out without the formation of an incision in a skin surface of the patient.

Such procedures are described in U.S. Pat. Nos. 5,297,536 and 5,458,131.

As described in those patents, a method for use in intra-abdominal surgery comprises the steps of (a) inserting an incising instrument with an elongate shaft through a natural body opening into a natural body cavity of a patient, (b) manipulating the incising instrument from outside the patient to form a perforation in an internal wall of the natural internal body cavity, and (c) inserting a distal end of an elongate surgical instrument through the natural body opening, the natural body cavity and the perforation into an abdominal cavity of the patient upon formation of the perforation. Further steps of the method include (d) inserting a distal end of an endoscope into the abdominal cavity, (e) operating the surgical instrument to perform a surgical operation on an organ in the abdominal cavity, (f) viewing the surgical operation via the endoscope, (g) withdrawing the surgical instrument and the endoscope from the abdominal cavity upon completion of the surgical operation, and (h) closing the perforation.

Visual feedback may be obtained as to position of a distal end of the incising instrument prior to the manipulating thereof to form the perforation. That visual feedback may be obtained via the endoscope or, alternatively, via radiographic or X-ray equipment.

The abdominal cavity may be insufflated prior to the insertion of the distal end of the endoscope into the abdominal cavity. Insufflation may be implemented via a Veress needle inserted through the abdominal wall or through another perforation in the internal wall of the natural body cavity. That other perforation is formed by the Veress needle itself. U.S. Pat. No. 5,209,721 discloses a Veress needle that utilizes ultrasound to detect the presence of an organ along an inner surface of the abdominal wall.

A method in accordance with the disclosures of U.S. Pat. Nos. 5,297,536 and 5,458,131 comprises the steps of (i) inserting an endoscope through a natural body opening into a natural body cavity of a patient, (ii) inserting an endoscopic type incising instrument through the natural body opening into the natural body cavity, (iii) manipulating the incising instrument from outside the patient to form a perforation in an internal wall of the natural internal body cavity, (iv) moving a distal end of the endoscope through the perforation, (v) using the endoscope to visually inspect internal body tissues in an abdominal cavity of the patient, (vi) inserting a distal end of an elongate surgical instrument into the abdominal cavity of the patient, (vii) executing a surgical operation on the internal body tissues by manipulating the surgical instrument from outside the patient, (viii) upon completion of the surgical operation, withdrawing the surgical instrument and the endoscope from the abdominal cavity, (ix) closing the perforation, and (x) withdrawing the endoscope from the natural body cavity.

The surgical procedures of U.S. Pat. Nos. 5,297,536 and 5,458,131 reduces trauma to the individual even more than laparoscopic procedures. Hospital convalescence stays are even shorter. There are some potential problems with the procedures, such as the difficulty in forming a fluid tight closure of the perforation formed in the wall of the hollow internal body organ. Certain intra-abdominal operations cannot be easily performed owing to the necessity or removing large chunks of organic or inorganic material (e.g., entire kidney, gall stones). Some operations can require the simultaneous usage of many different instruments so that space along the selected pathways may be difficult to find.

### OBJECTS OF THE INVENTION

It is an object of the present invention to provide improvements on the afore-described surgical procedures.

It is another object of the present invention to provide a method and/or an associated device for keeping a passageway open in an internal hollow organ.

These and other objects of the present invention will be apparent from the drawings and detailed descriptions herein. While every object of the invention is believed to be attained in at least one embodiment of the invention, there is not necessarily any single embodiment that achieves all of the objects of the invention.

### SUMMARY OF THE INVENTION

A surgical device in accordance with the present invention comprises a tubular member having a closure element provided at one end for effectuating a substantial closure of the tubular member at the one end. The closure element defines an aperture, while the tubular member has an effective outer diameter sufficiently small to enable disposition of the tubular member in a natural body recess, that is, a recess communicating with the ambient atmosphere via a natural body opening such as the vaginal orifice or the anus. The tubular member is provided with a clamping element at the one end for holding the tubular member to a web of organic tissue inside a patient. The web of organic tissue is a wall of an internal organ such as the colon or the vagina.

Preferably, the closure element is an end panel of the tubular member and the clamping element includes a disk member or a balloon member connected to the tubular member in parallel with the end panel so that the disk or balloon member cooperates with the end panel to clamp the web of organic tissue between the end panel and the disk or balloon member.

Where the clamping element is a disk member, the disk member is preferably made of a biocompatible flexible sheet or film material.

Preferably, the clamping element has an opening aligned with the aperture to enable insertion of a medical instrument through the aperture and the opening after disposition of the tubular member in a natural body recess of a patient so that the web of organic tissue is disposed between the end panel and the clamping element.

In accordance with another feature of the present invention, a seal in the form of a film or membrane of resilient material is connected at least indirectly to the disk and the closure element.

In accordance with a further feature of the present invention, at least one of the end panel and the clamping element is provided with a layer of a dormant adhesive substance that is activated by the application of a predetermined form of energy.

The end panel of the port device may be an inflatable member or a disk.

Pursuant to another feature of the present invention, the tubular member includes an inflatable balloon or bladder member having a substantially cylindrical inflated configuration.

In accordance with yet another feature of the present invention, the closure element is an end panel of the tubular member and the clamping element includes a depressurizable recess disposed in the end panel to face the web of organic tissue. The surgical device then further comprises means such as a tube or conduit operatively connected to the depressurizable recess for generating a partial vacuum therein.

In accordance with yet another feature of the present invention, an elongate tube extends longitudinally along the tubular member from an end opposite the one end of the tubular member and through the closure element so that the tube has two ends both disposed outside the tubular member. This tube is used for delivering gas to an internal space (e.g., abdominal cavity) to maintain pneumoperitoneum.

A surgical method in accordance with the present invention comprises (i) inserting a distal end portion of a surgical instrument through a natural body opening of a patient into a natural body cavity of the patient, (ii) using the surgical instrument to form a temporary artificial opening through a wall of an organ defining the natural body cavity, (iii) providing a surgical port device including a tubular member having a closure element provided at one end for effectuating a substantial closure of the tubular member at the one end, the closure element defining an aperture, (iv) inserting the port device through the natural body opening into the natural body cavity, (v) subsequent to the inserting of the port device, disposing the tubular member so that the aperture in the closure element is aligned with the artificial opening in the wall of the organ, (vi) also subsequent to the inserting of the port device, clamping the closure member and concomitantly the tubular member to the wall of the organ so that the aperture in the closure element remains aligned with the artificial opening in the wall of the organ, and (vii) after the clamping of the port device to the wall of the organ, inserting a distal end portion of a medical instrument through the natural body opening, the tubular member in the natural body cavity, the aperture, and the artificial opening into an internal space inside the patient.

The natural body cavity that receives the tubular surgical port device typically either the vagina or the colon.

Where the closure element is an end panel of the tubular member, the clamping of the port device to the wall of the organ includes disposing the wall of the organ between the end panel and a clamping member connected to the tubular member in parallel with the end panel. Pursuant to a feature of the present invention, the disposing of the wall between the end panel and the clamping member includes passing the clamping member in a collapsed configuration through the artificial opening and subsequently expanding the disk from the collapsed configuration to an expanded configuration.

Where the clamping member has an opening, the disposing of the wall between the end panel and the clamping member includes aligning the opening in the clamping member with the aperture to enable insertion of the medical instrument through the aperture and the artificial opening.

Where a seal is provided in the form of a film or membrane of resilient material, the inserting of the distal end portion of the medical instrument includes inserting the distal end portion of the instrument through the film or membrane so as to render the artificial opening with the medical instrument passing therethrough substantially air tight.

As indicated above, the clamping member of the method may be a disk or a balloon. In the case of a balloon, the disposing of wall of the organ between the end panel and the clamping member includes inflating the balloon.

Pursuant to an additional feature of the present invention, at least one of the end panel and the clamping member is provided with a layer of a dormant adhesive substance. The method then further comprises applying a predetermined form of energy to the layer of dormant adhesive substance after the disposing of wall of the organ between the end panel and the clamping member.

The end panel of the tubular may take the form of a balloon. In that case, the method further comprises inflating that balloon member after the inserting of the port device through the natural body opening into the natural body cavity.

Where the clamping member includes a depressurizable recess as described hereinabove, the clamping of the port device to the wall of the organ includes disposing the end panel adjacent the wall of the organ and subjecting the recess to an under-pressure to vacuum clamp the end panel to the wall of the organ.

As further described above, the port device may include an elongate tube extending longitudinally along the tubular member through the closure element. The method then additionally comprises delivering a gas under pressure to the internal space after the clamping of the port device to the wall of the organ and after the inserting of the distal end portion of the medical instrument into the internal space. This step maintains pneumoperitoneum in the internal space (e.g., abdominal cavity) of the patient.

A surgical kit comprises, in accordance with the present invention, a surgical instrument having a distal end insertable through a natural body opening of a patient into a natural body cavity of the patient. The surgical instrument is provided with an operative tip utilizable to form a temporary artificial opening through a wall of an organ defining the patient's natural body cavity. A surgical port device comprises a tubular member having a closure element provided at one end for effectuating a substantial closure of the tubular member at the one end, the closure element defining an aperture. The tubular member has an effective outer diameter sufficiently small to enable disposition of the tubular member in a natural body recess. The tubular member is provided with clamping means at the one end for holding the tubular member to a web of organic tissue inside a patient.

The device and method of the present invention serve to provide support to an inner wall of an internal hollow organ to facilitate the performance of a surgical or diagnostic operation via that hollow organ wall. The present invention makes it easier for a surgeon to locate and identify the artificial opening or aperture formed in the wall of the hollow organ, for purposes of inserting a succession of instruments through the artificial opening or aperture into the internal abdominal space of the patient. Pneumoperitoneum is also maintained.

### BRIEF DESCRIPTION OF THE DRAWINGS

FIG. 1 is a schematic longitudinal cross-sectional view of a surgical port device in accordance with the present invention, showing the device as used in a surgical procedure pursuant to the invention.

FIG. 2 is a partial schematic longitudinal cross-sectional view of the surgical port device of FIG. 1, showing additional details of the device.

FIG. 3 is a partial schematic longitudinal cross-sectional view similar to FIG. 2, showing a modified form of the surgical port device.

FIG. 4 is a partial schematic longitudinal cross-sectional view similar to FIGS. 2 and 3, showing another modified form of the surgical port device.

FIG. 5 is a partial schematic longitudinal cross-sectional view similar to FIGS. 2-4, showing a further modification of the surgical port device.

FIG. 6 is a partial schematic longitudinal cross-sectional view similar to FIGS. 2-5, showing an additional modification of the surgical port device.

FIG. 7 is a partial schematic longitudinal cross-sectional view similar to FIGS. 2-6, showing yet another modification of the surgical port device.

### DETAILED DESCRIPTION

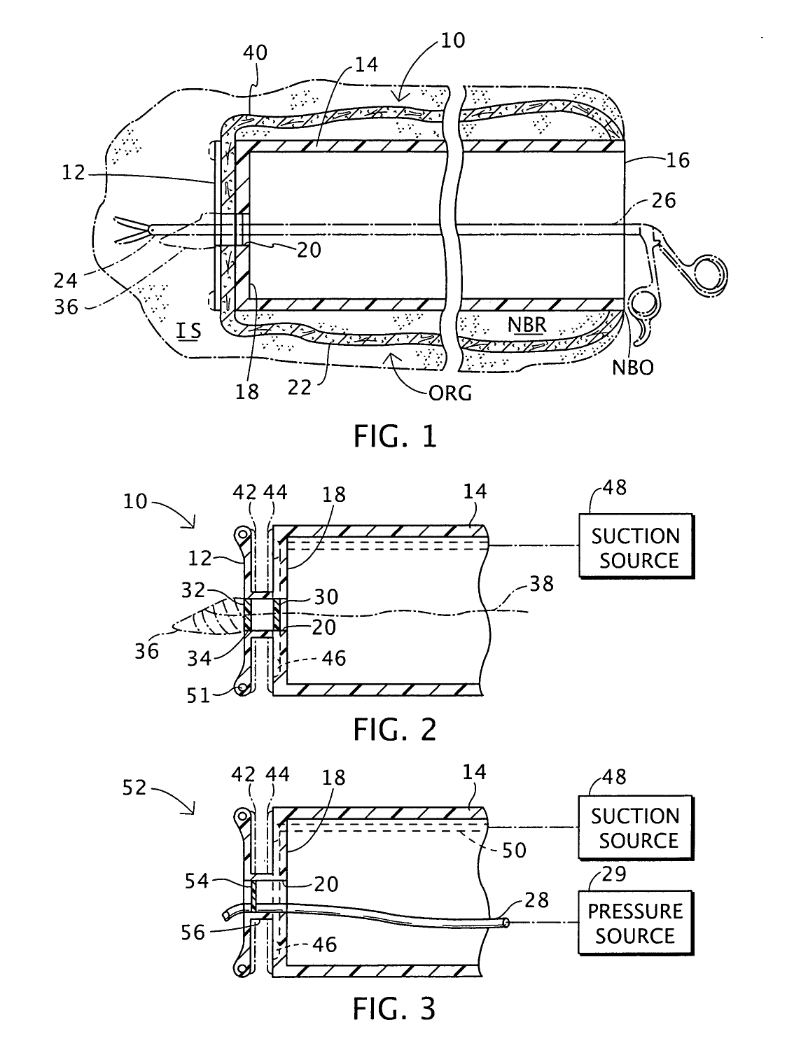

As shown in FIGS. 1 and 2, a surgical port device 10 for use in a trans-organ surgical procedure as described in U.S. Pat. Nos. 5,297,536 and 5,458,131 (both incorporated by reference herein) includes a disk 12 connected to a substantially rigid tubular member 14. Tubular member 14 is open at a proximal end 16, near a surgeon (not shown), and is provided at a distal end with a closure element 18 in the form of a disk-shaped end panel for effectuating a substantial closure of the tubular member at the distal end. Closure element 18 defines an aperture 20 smaller than an internal diameter (not designated) of tubular member 14. Tubular member 14 has an effective outer diameter (not designated) sufficiently small to enable disposition of the tubular member in a natural body cavity or recess NBR in an organ ORG such as the vagina or the sigmoidal colon, that communicates with the ambient atmosphere via a natural body opening NBO such as the vaginal orifice or the anus. Disk 12 functions as a clamping element at the distal end of tubular member 14 for holding the tubular member to a web 22 of organic tissue inside a patient. The web 22 of organic tissue is a wall of an internal organ such as the colon or the vagina.

In a medical procedure using the device of FIGS. 1 and 2, a surgeon inserts a distal end portion of a surgical instrument (not shown) through natural body opening NBO into natural body cavity NBR. This procedure is described in U.S. Pat. Nos. 5,297,536 and 5,458,131. The surgeon uses the surgical instrument to form a temporary artificial opening TAO through wall 20 of organ ORG. Surgical port device 10 is inserted through natural body opening NBO into natural body cavity NBR and then disposed so that aperture 20 in closure element or end panel 18 is aligned with artificial opening TAO in the wall of organ ORG. Also subsequent to the inserting of port device 10, closure member or end panel 18 and concomitantly tubular member 14 is clamped to wall 22 of organ ORG so that aperture 20 remains aligned with artificial opening TAO in the wall of the organ. Thereafter, the surgeon inserts a distal end portion 24 of a medical instrument 26 through natural body opening NBO, tubular member 14, located in the natural body cavity NBR, aperture 20, and artificial opening TAO into an internal space IS inside the patient.

Closure element or end panel 18 may be clamped to wall 22 of organ ORG solely by means of clamping disk 12. The clamping action of disk 12 may be enhanced by gas pressure (pneumoperitoneum) inside internal space IS. Disk 12 is pressed against wall 22 and closure element or end panel 18 by gas pressure inside space IS. Typically, internal space IS is the abdominal cavity, which is insufflated with carbon dioxide gas as described in U.S. Pat. Nos. 5,297,536 and 5,458,131. This pneumoperitoneum may be maintained during a surgical procedure by delivering carbon dioxide (or other gas) into the internal space via a tube 28 (see FIG. 3) that extends from a pressure source 29 outside the patient, along tubular member 14, and through closure element or end panel 18 and disk 12 into internal space IS. Pressurization tube 28 may be manufactured as a separate component that is attached to tubular member 14. Alternatively, pressurization tube 28 may be formed at least partially as a channel or bore within the wall of tubular member 14. In is contemplated that pressurization tube 28 extends through aperture 20 in closure element or end panel 18. Thus, tube 28 passes through artificial opening TAO when port device 10 is properly deployed in natural body cavity NBR for assisting in the performance of a surgical operation at a site within internal space IS via wall 22 of organ ORG.

As illustrated in FIG. 2, port device 10 includes a valve element 30 in the form of a self-sealing membrane or film attached to closure element or end panel 18 at aperture 20. A second valve element 32 also in the form of a self-sealing membrane or film may be attached to disk 12 in alignment with aperture 20 and valve element 30. Valve elements 30 and 32 are made of a resilient polymeric material that may be formed centrally with a pin-hole or perforation for the passage of distal end portion 24 of medical instrument 26. Valve elements 30 and 32 cooperate with the shaft (not separatedly designated) of medical instrument 26 to effectuate an air-tight closure about the medical instrument. Valve element or membrane 32 is located in an aperture or opening 34 centrally located in disk 12.

As illustrated in FIGS. 1 and 2, disk 12 may be originally disposed in a collapsed, wound or folded configuration 36 facilitating insertion of the disk through artificial opening TAO. To that end, disk is made of a biocompatible flexible sheet material. Collapsed disk configuration 36 may surround a distal end portion of the incising instrument, as shown in FIG. 1 with respect to medical instrument 26. In that case, the incising instrument is inserted into natural body cavity NBR together with surgical port device 10 and manipulated to cut wall 22 while the instrument traverses the port device. Collapsed disk configuration 36 is inserted through opening TAO, possibly while the collapsed configuration 36 surrounds the incising instrument shaft. After passing of collapsed disk configuration 36 through opening TAO, the surgeon may pull a string 38 extending to the collapsed disk configuration 26, to thereby release the disk 12 and enable it to expand and become spread out against an outer surface 40 of organ wall 22. The organ wall 22 is then sandwiched or clamped between the disk 12 and closure element or end panel 18.

Disk 12 extends generally parallel to and spaced from end panel 18 of tubular member 14. Disk 12 may be provided on a surface (not separately designated) facing end panel 18 with a layer 42 of a dormant adhesive substance that is activated by the application of a predetermined form of energy such as infrared radiation, heat energy, electrical current, or ultrasonic pressure waves. Alternatively or additionally, end panel 18 may be provided on a surface facing disk 12 with a layer 44 similarly of a dormant adhesive substance that is activated by the application of such energy. In the latter case, the activation energy is applied after adhesive layer 44 has been placed into suitable contact with organ wall 22, after or possibly before artificial opening TAO has been incised. In the former case, the activation energy is applied after disk 12 has been spread out so that adhesive layer 42 is in contact with surface 40 of wall 22.

As shown in FIG. 2, end panel 18 may be provided in a surface facing disk 12 with an annular groove or recess 46 that is connectable to a vacuum or suction source 48 (FIG. 3) via a channel or conduit 50 in tubular member 14. After end panel 18 has been placed into position in contact with organ wall 22, source 48 may be activated (for instance, by opening a valve—not shown) to subject groove 46 to at least a partial vacuum to hold port device 10 against the organ wall. The depressurization of groove 46 forms a seal around artificial opening TAO not only to assist in holding port device 10 in place but also to prevent the leaking of contaminants into internal space IS via artificial opening TAO.

Disk 12 is optionally provided along an outer periphery with a ring 51 embedded in the flexible polymeric material of the disk. Ring 51, although flexible, is stiffer than the sheet or film material of disk 12 and thus serves as a spring to maintain disk 12 in an expanded configuration.

A surgical port device 52 depicted in FIG. 3 is identical to surgical port device 10 of FIGS. 1 and 2, except that valve elements or membranes 30 and 32 have been replaced with a single valve element 54 having the same structure and formed of the same material as valve elements 30 and 32. Disk 12 and end panel 18 are connected to one another via a tubular sleeve 56 that defines an aperture through disk 12 and end panel 18. Sleeve 56 may be made of the same substantially rigid polymeric material as tubular member 14 and may be integrally formed therewith. Valve element or membrane 54 extends across the lumen (not separately designated) of sleeve 56 to assist in sealing internal space IS (FIG. 1) against the loss of pneumoperitoneum while permitting access to that sace by medical instrument 26.

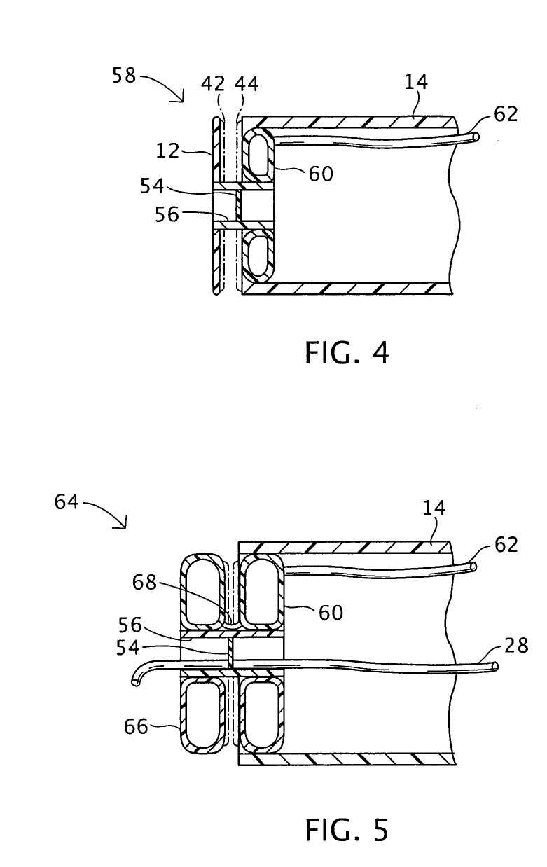

A surgical port device 58 illustrated in FIG. 4 is identical to surgical port device 52 of FIG. 3, except that end panel 18 has been replaced with an annular balloon member 60. Balloon member 60 is connected about an outer periphery to tubular member 14 and about an inner periphery to tubular sleeve 56. Balloon member 60 is inflatable by the introduction of a fluid such as saline solution, air or carbon dioxide gas via a tube 62. Balloon 60 is inflated to exert a clamping pressure against disk 12, thereby firmly holding the port device 58 to organ wall 22. Tube 62 is depicted as a separate tube extending into tubular member 14. However, tube 62 may be realized as a channel or bore in the cylindrical sidewall of tubular member 14.

A surgical port device 64 illustrated in FIG. 5 is identical to surgical port device 58 of FIG. 4, except that disk 12 has been replaced with an annular balloon member 66. Balloon member 66 is connected along an inner periphery to tubular sleeve 56. Balloon member 66 is inflatable by the introduction of a fluid such as saline solution, air or carbon dioxide gas via tube 62, balloon member 60 and a shunt conduit 68 extending along tubular sleeve 56. Balloon members 62 and 66 are inflated to exert a clamping pressure on one another to thereby firmly hold the port device 64 to organ wall 22. Balloon members 62 and 64 are depicted in a partially inflated configuration in FIG. 5 to illustrate adhesive layers 42 and 44. Pressurization tube 28 extends through valve element or membrane 53.

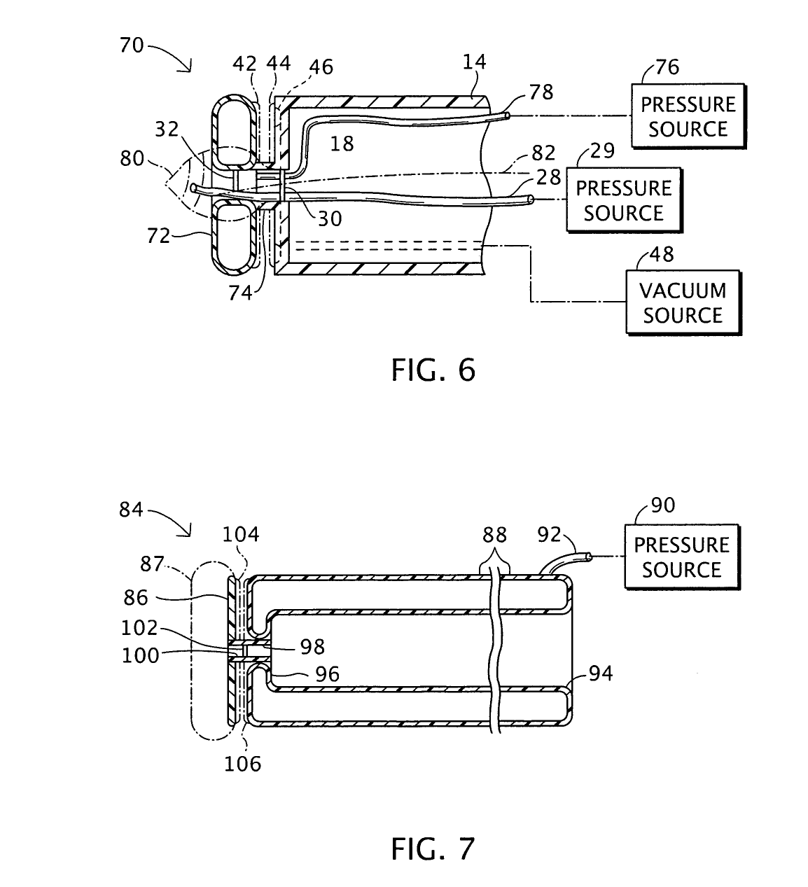

A surgical port device 70 illustrated in FIG. 6 is identical to surgical port device 10 of FIGS. 1 and 2, except that disk 12 has been replaced with an annular balloon member 72. Balloon member 72 is connected along an inner periphery to a tubular sleeve 74. Balloon member 72 is inflatable by the introduction of a fluid such as saline solution, air or carbon dioxide gas from a pressure source 76 via a tube 78 that extends along sleeve 74 and through valve element or membrane 30. Balloon member 72 is inflated to exert a clamping pressure on end panel 18 to thereby firmly hold the port device 70 to organ wall 22. Balloon member 72 is depicted in a partially inflated configuration in FIG. 5 to illustrate adhesive layers 42 and 44.

As illustrated in FIG. 6, balloon 72 may be originally disposed in a collapsed, wound or folded configuration 80 facilitating insertion of the balloon through artificial opening TAO. Collapsed balloon configuration 80 may surround a distal end portion of the incising instrument during an initial phase of a surgical operation as disclosed herein. The incising instrument is used to form artificial opening TAO and to guide collapsed balloon configuration 80 through the incised opening. After passing of collapsed balloon configuration 80 through opening TAO, the surgeon may pull a string 82 extending to the collapsed balloon 80, to thereby release the balloon and enable it to expand and become inflated via tube 78 to spread out against outer surface 40 of organ wall 22. This same discussion as to collapse balloon configuration 80 applies to the use of port device 64 of FIG. 5.

As shown in FIG. 7, a surgical port device 84 for use in a trans-organ surgical procedure as described in U.S. Pat. Nos. 5,297,536 and 5,458,131 includes a disk 86 or balloon 87 connected to a tubular member 88 that is implemented as an inflatable bladder or balloon connectable to a pressure source 90 via a tube 92. Tubular member 88 is open at a proximal end 94, near a surgeon (not shown), and is formed at a distal end with a closure portion 96 in the form of an inflatable end panel for effectuating a substantial closure of the tubular member at the distal end. Tubular bladder or balloon member 88 has a substantially cylindrical inflated configuration.

Closure portion 96 defines an aperture 98 smaller than an internal diameter (not designated) of tubular member 88. Tubular member 88 has an effective outer diameter (not designated) sufficiently small to enable disposition of the tubular member in a natural body cavity or recess NBR (FIG. 1) in an organ ORG (FIG. 1) such as the vagina or the sigmoidal colon, that communicates with the ambient atmosphere via a natural body opening NBO (FIG. 1) such as the vaginal orifice or the anus. Disk 86 or balloon 87 functions as a clamping element at the distal end of tubular member 88 for holding the tubular member to a web 22 (FIG. 1) of organic tissue inside a patient. The web 22 of organic tissue is a wall of internal organ ORG, exemplarily the colon or the vagina.

In a medical procedure using the device of FIG. 7, a surgeon cuts temporary artificial opening TAO as described in U.S. Pat. Nos. 5,297,536 and 5,458,131. Surgical port device 84 is inserted in a deflated configuration through natural body opening NBO into natural body cavity NBR and subsequently inflated via tube 92. The inflated tubular member 88 is so disposed that aperture 98 in closure portion 96 is aligned with artificial opening TAO in the wall of organ ORG. An inner periphery of closure portion 96 along aperture 98 is attached to a tubular sleeve 100 to which disk 86 or balloon 87 is also attached. Sleeve 100 is aligned with and inserted through the artificial opening TAO in organ wall 22.

The deployment of port device 84 includes inserting disk 86 or balloon 87 in a folded or furled configuration through artificial opening TAO and then opening the disk or balloon to enable an expansion thereof against organ wall 22 in opposite to pressure applied by the pressurization of tubular member 88 and more particularly closure portion 96. Thereafter, the surgeon inserts a distal end portion 24 of a medical instrument 26 (FIG. 1) through natural body opening NBO, tubular member 88, located in the natural body cavity NBR, aperture 98, and artificial opening TAO into internal space IS.

The clamping action of disk 86 or balloon 87 may be enhanced by gas pressure (pneumoperitoneum) inside internal space IS. Disk 86 or balloon 87 is pressed against wall 22 and closure portion 96 by gas pressure inside space IS. Typically, internal space IS is the abdominal cavity, which is insufflated with carbon dioxide gas as described in U.S. Pat. Nos. 5,297,536 and 5,458,131. This pneumoperitoneum may be maintained during a surgical procedure by delivering carbon dioxide (or other gas) into the internal space via a tube 28 (FIG. 3) that extends from a pressure source 29 outside the patient, along tubular member 88, and through closure portion 96 and disk 86 or balloon 87 into internal space IS.

As further illustrated in FIG. 7, port device 84 includes a valve element 102 in the form of a self-sealing membrane or film disposed in and attached to sleeve 100. Valve element 102 is made of a resilient polymeric material that may be formed centrally with a pin-hole or perforation for the passage of distal end portion 24 of medical instrument 26.

The operation and use of disk 86 or balloon 87 are similar to that of disk 12 (FIG. 2) or balloon member 72 (FIG. 6). Accordingly, disk 86 or balloon 87 may be provided on a surface (not separately designated) facing closure portion 96 with a layer 104 of a dormant adhesive substance that is activated by the application of a predetermined form of energy such as infrared radiation, heat energy, electrical current, or ultrasonic pressure waves. Alternatively or additionally, closure portion 96 may be provided on a surface facing disk 86 with a layer 106 similarly of a dormant adhesive substance that is activated by the application of such energy. In the latter case, the activation energy is applied after adhesive layer 106 has been placed into suitable contact with organ wall 22, after or possibly before artificial opening TAO has been incised. In the former case, the activation energy is applied after disk 86 or balloon has been spread out so that adhesive layer 104 is in contact with surface 40 (FIG. 1) of wall 22.

Port device 10 may be distributed by itself or in a kit in combination with one or more medical instruments 26, including scalpels, forceps, scissors, cauterizers, snares, retrieval bags, etc.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are profferred by way of example to facilitate comprehension of the invention and should not be construed to limit the scope thereof.

What is claimed is:

1. A surgical device comprising a tubular member having a closure element provided at one end for effectuating a substantial closure of said tubular member at said one end, said closure element defining an aperture, said tubular member having an effective outer diameter sufficiently small to enable disposition of said tubular member in a natural body recess, said tubular member being provided with clamping means at said one end for holding said tubular member to a web of organic tissue inside a patient.

2. The surgical device defined in claim 1 wherein said closure element is an end panel of said tubular member and wherein said clamping means includes a disk member connected to said tubular member in parallel with said end panel so that said disk member cooperates with said end panel to clamp the web of organic tissue between said end panel and said disk member.

3. The surgical device defined in claim 2 wherein said disk member is made of a flexible sheet material.

4. The surgical device defined in claim 3 wherein said disk member has an opening aligned with said aperture to enable insertion of a medical instrument through said aperture and said opening after disposition of said tubular member in a natural body recess of a patient so that the web of organic tissue is disposed between said end panel and said disk member.

5. The surgical device defined in claim 4, further comprising a seal in the form of a film or membrane of resilient material attached at least indirectly to said disk and said closure element.

6. The surgical device defined in claim 2 wherein at least one of said end panel and said disk is provided with a layer of a dormant adhesive substance that is activated by the application of a predetermined form of energy.

7. The surgical device defined in claim 2 wherein said end panel is taken from the group consisting of an inflatable member and a disk element.

8. The surgical device defined in claim 1 wherein said closure element is an end panel of said tubular member and wherein said clamping means includes a balloon member connected to tubular member so that said balloon member when inflated is disposed in parallel with said end panel to clamp said web of organic tissue between said end panel and said balloon member.

9. The surgical device defined in claim 8 wherein said balloon member defines an opening aligned with said aperture to enable insertion of a medical instrument through said aperture and said opening after disposition of said tubular member in a natural body recess of a patient so that the web of organic tissue is disposed between said end panel and said balloon member.

10. The surgical device defined in claim 9, further comprising a seal in the form of a film or membrane of resilient material connected at least indirectly to said balloon and said end panel.

11. The surgical device defined in claim 8 wherein at least one of said end panel and said balloon member is provided with a layer of a dormant adhesive substance that is activated by the application of a predetermined form of energy.

12. The surgical device defined in claim 8 wherein said end panel is taken from the group consisting of an inflatable member and a disk member.

13. The surgical device defined in claim 1, further comprising a valve element coupled to said tubular member for forming a seal about an instrument shaft inserted through said aperture.

14. The surgical device defined in claim 1 wherein said tubular member includes an inflatable balloon or bladder member having a substantially cylindrical inflated configuration.

15. The surgical device defined in claim 1 wherein said closure element is an end panel of said tubular member and wherein said clamping means includes a depressurizable recess disposed in said end panel to face said web of organic tissue, further comprising means operatively connected to said depressurizable recess for generating a partial vacuum therein.

16. The surgical device defined in claim 1, further comprising an elongate tube extending longitudinally along said tubular member from an end opposite said one end and through said closure element so that said tube has two ends both disposed outside said tubular member.

17. A surgical method comprising:
providing a surgical port device including a tubular member having a closure element provided at one end for effectuating a substantial closure of said tubular member at said one end, said closure element defining an aperture;
inserting said port device through a natural body opening of a patient into a natural body cavity;
inserting a distal end portion of a surgical instrument through said natural body opening into said natural body cavity;
using said surgical instrument to form a temporary artificial opening through a wall of an organ defining said natural body cavity;
subsequent to the inserting of said port device, disposing said tubular member so that said aperture in said closure element is aligned with said artificial opening in said wall of said organ;
also subsequent to the inserting of said port device, clamping said closure member and concomitantly said tubular member to said wall of said organ so that said aperture in said closure element remains aligned with said artificial opening in said wall of said organ; and
after the clamping of said port device to said wall of said organ, inserting a distal end portion of a medical instrument through said natural body opening, said tubular member in said natural body cavity, said aperture, and said artificial opening into an internal space inside the patient.

18. The surgical method defined in claim 17 wherein said closure element is an end panel of said tubular member and wherein the clamping of said port device to said wall of said organ includes disposing said wall of said organ between said end panel and a clamping member connected to said tubular member in parallel with said end panel.

19. The surgical method defined in claim 18 wherein the disposing of said wall between said end panel and said clamping member includes passing said clamping member in a collapsed configuration through said artificial opening and subsequently expanding said disk from said collapsed configuration to an expanded configuration.

20. The surgical method defined in claim 18 wherein said clamping member has an opening, the disposing of said wall between said end panel and said clamping member including aligning the opening in said clamping member with said aperture to enable insertion of said medical instrument through said aperture and said artificial opening.

21. The surgical method defined in claim 18 wherein at least one of said clamping member and said end panel is provided with a seal in the form of a film or membrane of resilient material, the inserting of said distal end portion of said medical instrument including inserting said distal end portion through said film or membrane so as to render said artificial opening with said medical instrument passing therethrough substantially air tight.

22. The surgical method defined in claim 18 wherein said clamping member is taken from the group consisting of a disk and a balloon.

23. The surgical method defined in claim 18 wherein at least one of said end panel and said clamping member is provided with a layer of a dormant adhesive substance, further comprising applying a predetermined form of energy to said layer of dormant adhesive substance after the disposing of wall of said organ between said end panel and said clamping member.

24. The surgical method defined in claim 18 wherein said end panel is a balloon member, further comprising inflating said balloon member after the inserting of said port device through said natural body opening into said natural body cavity.

25. The surgical method defined in claim 17 wherein said closure element is an end panel of said tubular member and wherein said clamping means includes a depressurizable recess, the clamping of said port device to said wall of said organ includes disposing said end panel adjacent said wall of said organ and subjecting said recess to an under-pressure to vacuum clamp said end panel to said wall of said organ.

26. The surgical method defined in claim 17, wherein said port device includes an elongate tube extending longitudinally along said tubular member through said closure element, further comprising delivering a gas under pressure to said internal space after the clamping of said port device to said wall of said organ and after the inserting of said distal end portion of said medical instrument into said internal space.

27. A surgical kit comprising:
a surgical instrument having a distal end insertable through a natural body opening of a patient into a natural body cavity of the patient, said surgical instrument being provided with an operative tip utilizable to form a temporary artificial opening through a wall of an organ defining the patient's natural body cavity;
a surgical port device comprising a tubular member having a closure element provided at one end for effectuating a substantial closure of said tubular member at said one end, said closure element defining an aperture, said tubular member having an effective outer diameter sufficiently small to enable disposition of said in a natural body recess, said tubular member being provided with a clamping means at said one end for holding said tubular member to a web of organic tissue inside a patient.
